SEQUENCE LISTING (14 sequences)

SEQ ID NO: 1 - DNA, 20 bp, Artificial Sequence
  mouse type I collagen alpha chain forward primer
acgtcctggtgaagttggtc

SEQ ID NO: 2 - DNA, 22 bp, Artificial Sequence
  mouse type I collagen  alpha chain reverse primer
ggaagcctctctctcctctctg

SEQ ID NO: 3 - DNA, 20 bp, Artificial Sequence
  mouse elastin forward primer
gaggtttagtgcctggtgga

SEQ ID NO: 4 - DNA, 20 bp, Artificial Sequence
  mouse elastin reverse primer
cacctctggctccgtatttg

SEQ ID NO: 5 - DNA, 20 bp, Artificial Sequence
  mouse MMP13 forward primer
tggaccttctggtcttctgg

SEQ ID NO: 6 - DNA, 20 bp, Artificial Sequence
  mouse MMP13 reverse primer
tcatgggcagcaacaataaa

SEQ ID NO: 7 - DNA, 20 bp, Artificial Sequence
  28s rRNA forward primer
acggtaacgcaggtgtccta

SEQ ID NO: 8 - DNA, 20 bp, Artificial Sequence
  28s rRNA reverse primer
ccgctttcacggtctgtatt

SEQ ID NO: 9 - DNA, 20 bp, Artificial Sequence
  human type I collagen alpha chain forward primer
acgtcctggtgaagttggtc

SEQ ID NO: 10 - DNA, 22 bp, Artificial Sequence
  human type I collagen alpha chain reverse primer
ggaagcctctctctcctctctg

SEQ ID NO: 11 - DNA, 20 bp, Artificial Sequence
  human elastin forward primer
gagtgaagcctgggaaagtg

SEQ ID NO: 12 - DNA, 20 bp, Artificial Sequence
  human elastin reverse primer
actcctgctccagtgggaac

SEQ ID NO: 13 - DNA, 20 bp, Artificial Sequence
  human MMP1 forward primer
ccaaggtctctgagggtcaa

SEQ ID NO: 14 - DNA, 20 bp, Artificial Sequence
  human MMP1 reverse primer
caagatttcctccaggtcca

(19) United States
(12) Patent Application Publication
(10) Pub. No.: US 2012/0149775 A1
(43) Pub. Date: Jun. 14, 2012

(54) METHODS FOR PREVENTING AND IMPROVING SKIN ELASTIC PROPERTY LOSS

(75) Inventor: Tomonobu Ezure, Yokohama-shi (JP)

(73) Assignee: SHISEIDO COMPANY, LTD., TOKYO (JP)

(21) Appl. No.: 13/306,478

(22) Filed: Nov. 29, 2011

Related U.S. Application Data
(60) Provisional application No. 61/417,638, filed on Nov. 29, 2010.

(51) Int. Cl. A61K 31/202; A61P 17/02; A61Q 19/08; A61K 8/36

(52) U.S. Cl. 514/560

(57) ABSTRACT

The present invention is directed to method for preventing and improving an aggravation of a skin condition accompanied with a skin elastic property loss by suppressing an increase in, or inhibiting the physiological activity of, palmitic acid. The present invention provides a cosmetic method for preventing and improving wrinkling and sagging accompanied with a skin elastic property loss and a method for preventing and treating a wound healing disorder and a bedsore.

### CROSS REFERENCE TO RELATED APPLICATIONS

The present application claims priority under 35 U.S.C. §119 to U.S. Provisional Patent Application No. 61/417,638, filed Nov. 29, 2010, the contents of which are hereby incorporated herein by reference.

### FIELD OF THE INVENTION

The present invention relates to a method for preventing and/or improving a skin elastic property loss and a method for preventing and/or improving a reduction in an extracellular matrix component.

### BACKGROUND ART

Skin is comprised of epidermis, dermis, subcutaneous tissue and the like. The skin also serves as a supportive tissue which supports the inside of a body, and its physical properties are important for defending against an external physical stimulation, keeping internal tissues in place and so on. Deterioration in such physical properties of the skin, especially skin viscoelasticity, for some reason, is known to cause an aggravation of sagging of the skin (Non-Patent Document 1).

We already showed that, in a dermis layer with expanded subcutaneous fat, a matrix metalloproteinase (MMP) is increased to reduce the number of fibroblasts, and that a hypertrophic fat cell suppresses not only the fibroblast proliferation but also the production of extracellular matrix components (collagen, elastin and hyaluronic acid) by the fibroblasts (Patent Document 1). We also reported that the facial skin elastic property has a statistically significant negative correlation with the subcutaneous fat layer thickness, and the extracellular matrix component is reduced as the subcutaneous fat is increased, resulting in a reduction in the viscoelasticity of the skin (Patent Document 1).

### PRIOR ART DOCUMENTS

### Non-Patent Documents

Non-Patent document 1: Ahn, S. et al., Skin Research and Technology, 13:280-284.

### Patent Documents

Patent document 1: PCT/JP2010/056617 Specification

### DISCLOSURE OF THE INVENTION

### Problem to be Solved by the Invention

Retinoids or vitamin A derivatives have been conventionally employed for improving skin elastic property loss. However, the retinoids have inflammatory side effects known as retinoid reactions. On the other hand, for the purpose of promoting fibroblast proliferation, Fibroblast Growth Factor, or a compound which activates a signal transmission involving the growth factor is also employed. Since each of them has effects on a wide range of factors, promoting the production of extracellular matrix components and the fibroblast proliferation as well as affecting vital functions substantially, they cannot be used safely on a daily basis. Accordingly, there is a need to develop a daily-usable, stable and safe composition which is a composition capable of preventing and improving a skin elastic property loss.

We new find that an increase of palmitic acid suppresses the fibroblast proliferation and the gene expression of collagen and elastic, while the matrix metalloproteinase (MMP) gene expression is augmented. We also find that eicosapentaenoic acid (EPA) alleviates fibroblast proliferation suppression by palmitic acid and by a fat cell. These findings demonstrate that the skin elastic property loss is attributable to an increase in palmitic acid. Furthermore, it demonstrates that the suppression of palmitic acid increase and inhibition of physiological activities can alleviate the suppression of fibroblast proliferation and the suppression of gene expression of collagen, elastin and the like.

### Means for Solving the Problem

The present invention provides a method for preventing and/or improving a skin elastic property loss, comprising a step of suppressing an increase in palmitic acid or a step of inhibiting the physiological activity of palmitic acid.

In the method for preventing and/or improving a skin elastic property loss according to the present invention, the step of suppressing an increase in palmitic acid may comprise a step of administering a composition which suppresses an increase in palmitic acid to a subject.

In the method for preventing and/or improving a skin elastic property loss according to the present invention, the step of inhibiting the physiological activity of palmitic acid may comprise a step of administering a composition which inhibits the physiological activity of palmitic acid to a subject.

In the method for preventing and/or improving a skin elastic property loss according to the present invention, the composition which inhibits the physiological activity of palmitic acid may be a composition comprising one or two or more of compounds selected from the group consisting of a polyvalent unsaturated fatty acid and a derivative and/or a salt thereof.

In the method for preventing and/or improving a skin elastic property loss according to the present invention, the polyvalent unsaturated fatty acid may be eicosapentaenoic acid.

The present invention provides a cosmetic method for preventing and/or improving wrinkling and sagging accompanied with a skin elastic property loss. The method comprises a step of applying to a skin a method for preventing and/or improving a skin elastic property loss according to the present invention.

The present invention provides a method for preventing and/or treating a wound healing disorder and a bedsore accompanied with a skin elastic property loss. The method comprises a step of applying to a skin a method for preventing and/or improving a skin elastic property loss according to the present invention.

The present invention provides a method for preventing and/or improving a reduction in an extracellular matrix component, comprising a step of suppressing an increase in palmitic acid or a step of inhibiting the physiological activity of palmitic acid.

In the method for preventing and/or improving a reduction in an extracellular matrix component according to the present invention, the step of suppressing an increase in palmitic acid may comprise a step of administering a composition which suppresses an increase in palmitic acid to a subject.

In the method for preventing and/or improving a reduction in an extracellular matrix component according to the present invention, the step of inhibiting the physiological activity of palmitic acid may comprise a step of administering a composition which inhibits the physiological activity of palmitic acid to a subject.

In the method for preventing and/or improving a reduction in an extracellular matrix component according to the present invention, the composition which inhibits the physiological activity of palmitic acid may be a composition comprising one or two or more of compounds selected from the group consisting of a polyvalent unsaturated fatty acid and a derivative and/or a salt thereof.

In the method for preventing and/or improving a reduction in an extracellular matrix component according to the present invention, the polyvalent unsaturated fatty acid may be eicosapentaenoic acid.

In the method for preventing and/or improving a reduction in an extracellular matrix component according to the present invention, the extracellular matrix component may be a collagen and/or an elastic. The collagen may be a type I collagen α chain.

The present invention provides a cosmetic method for preventing and/or improving wrinkling and sagging. The method comprises a step of applying to a skin a method for preventing and/or improving a reduction in an extracellular matrix component according to the present invention.

The present invention provides a method for preventing and/or treating a wound healing disorder and a bedsore. The method comprises a step of applying to a skin a method for preventing and/or improving a reduction in an extracellular matrix component according to the present invention.

In the method for preventing and/or improving a skin elastic property loss and the method for preventing and/or improving a reduction in an extracellular matrix component according to the present invention, the palmitic acid may be produced by a hypertrophic fat cell.

The present invention provides a composition for preventing and/or improving an aggravation of a skin condition accompanied with a skin elastic property loss, comprising a composition which suppresses an increase in palmitic acid, or inhibits the physiological activity of palmitic acid.

As used herein, a “wrinkle” refers to a kind of skin trouble, and is a state in which patterns formed from linear recesses on the skin surface are concentrated on a particular region where they exist with an irregularity in size and alignment.

As used herein, a “sag” refers to a kind of skin trouble, and is a state in which the skin tension is lost and a swollen skin is observed on the entire face including a periocular region, a perioral region, and lower cheeks, jaw, neck and the like.

As used herein, a “bedsore” refers to a skin ulcer formed as a result of a necrosis of skin and subcutaneous tissues in which the blood flow was blocked focally due to a pressure attributable to subject's own body weight.

As used herein, a “wound healing disorder” refers to a condition in which a physiological process for repairing a damaged site is not implemented normally or is suppressed. Such a physiological process includes, but is not limited to, immune response, angiogenesis, apoptosis, cell migration, cell proliferation, proliferation factor production, and fibroblast extracellular matrix component production.

As used herein, a “matrix metalloproteinase (MMP)” refers to an enzyme belonging to MMP family which binds to a metal, especially to zinc, and has an activity for cleaving most of the extracellular matrix constituents. Twenty-five or more of MMP family member enzymes have been identified. The amino acid and polynucleotide sequences of an MMP family member enzyme can be searched in the United State NCBI website (http://www.ncbi.nlm.nih.gov/sites/gquery) which contains OMIM (registered trademark, Online Mendelian Inheritance in Man) database. Among the MMP family members, human MMP1 mainly break down type I, type II and type III collagens. Human MMP2 and MMP9 break down elastin. Human MMP1 and mouse MMP13 are homologous genes.

A step of suppressing an increase in palmitic acid according to the present invention can be accomplished by various means including, but not limited to, administration of a composition which suppresses an increase in a subcutaneous fat to a subject. Such means may be accomplished in combination with each other.

A composition which suppresses an increase in palmitic acid according to the present invention includes, but is not limited to, an adiposity inhibitor, a lipid synthesis inhibitor, an anorectic agent, a fat cell differentiation inhibitor, a fat cell proliferation inhibitor, a fat metabolism modifier and the like. The adiposity-inhibitor includes, but is not limited to, a lipase inhibitor produced in a pancreas (Japanese Unexamined Patent Application Publication No. 2001-226274), an elastase and the like which promote degradation and excretion of hepatic and blood triglycerides. The lipid synthesis inhibitor includes, but is not limited to, an HMG-CoA reductase inhibitor such as pravastatin sodium, and a fibrate-based agent which acts on an intranuclear receptor PPAR-α to control the synthesis of a protein involved in a lipid synthesis. The anorectic agent includes, but is not limited to, mazindol, leptin and the like. The fat cell differentiation inhibitor includes, but is not limited to, the extracts of madder plant, sweet hydrangea leaf and the like (Japanese Unexamined Patent Application Publication No. 2002-138014). The fat cell proliferation inhibitor includes, but is not limited to, dihomo-γ-linolenic acid (Japanese Unexamined Patent Application Publication No. 2006-306813). The fat metabolism modifier includes, but is not limited to, a thiazolidine-based insulin sensitizer or the like such as pioglitazone.

A step of inhibiting the physiological activity of palmitic acid according to the present invention can be accomplished by various means including, but not limited to, administration of a composition which inhibits the physiological activity of palmitic acid to a subject. The mechanism of inhibiting the physiological activity of palmitic acid includes, but is not limited to, an antagonism. The step of inhibiting the physiological activity of the palmitic acid may be applied to a subject in combination with a step of suppressing an increase in the palmitic acid.

A composition which inhibits the physiological activity of palmitic acid according so the present invention includes, but is not limited to, a composition comprising one or two or more of compounds selected from the group consisting of a polyvalent unsaturated fatty acid and a derivative and/or a salt thereof. The composition may be a food product. The polyvalent unsaturated fatty acid may be eicosapentaenoic acid (EPA). The composition may be employed alone or in combination with other compositions.

In the present invention, measurements of the number of fibroblasts, and the expression levels of genes constituting extracellular matrix and the matrix metalloproteinase genes may be carried out using any measuring methods known to those skilled in the cosmetic and medical arts.

All references cited herein are hereby incorporated by reference in their entirety.

### BRIEF DESCRIPTION OF THE DRAWINGS

FIG. 1 is a bar graph showing the effect of palmitic acid on the proliferation of a mouse fibroblast.

FIG. 2 is a bar graph showing the effect of oleic acid on the proliferation of a mouse fibroblast.

FIG. 3 is a bar graph showing the effect of stearic acid on the proliferation of a mouse fibroblast.

FIG. 4 is a bar graph showing the effect of palmitic acid on the type I collagen α chain gene expression of a mouse fibroblast.

FIG. 5 is a bar graph showing the effect of palmitic acid on the elastin gene expression of a mouse fibroblast.

FIG. 6 is a bar graph showing the effect of palmitic acid on the matrix metalloproteinase 13 gene expression of a mouse fibroblast.

FIG. 7 is a bar graph showing the effect of palmitic acid on the proliferation of a human fibroblast.

FIG. 8 is a bar graph showing the effect of palmitic acid on the type I collagen α chain gene expression of a human fibroblast.

FIG. 9 is a bar graph showing the effect of palmitic acid on the elastin gene expression of a human fibroblast.

FIG. 10 is a bar graph showing the effect of palmitic acid on the matrix metalloproteinase 1 gene expression of a human fibroblast.

FIG. 11 is a bar graph showing the effect of palmitic acid and eicosapentaenoic acid on the proliferation of a fibroblast.

FIG. 12 is a bar graph showing the effect of a fat cell and eicosapentaenoic acid on the proliferation of a fibroblast during culturing together.

### DESCRIPTION OF EMBODIMENTS

Examples of the present invention described below are intended only to exemplify the invention rather than to limit the scope thereof. The scope of the present invention is limited only by the description in claims. Any change in the present invention, such as addition, deletion and substitution of a subject matter of the invention, is allowed without departing from the scope of the present invention.

### Example 1

### Mouse Fibroblast Proliferation Suppressing Effect of Palmitic Acid

### 1. Materials and Methods

### (1) Cell Culture

The cell employed was a mouse 3T3-L1 cell. This cell was inoculated. at 3×10⁴ cells/mL to a commercially available 24-well culture plate (353047, FALCON, Becton, Dickinson and Company Japan), where it was cultured in a commercially available cell culture medium (D-MEM, 11885084, GIBCO, Life Technologies Japan Ltd.) supplemented with 10% bovine serum (16010159, GIBCO, Life Technologies Japan Ltd.). The cell was cultured for about 6 hours in 5% CO₂ and saturated water vapor atmosphere at 37° C.

Thereafter, the culture medium for culturing the mouse fibroblast was switched to a medium which was the above-mentioned cell culture medium supplemented with 0.5% bovine serum (hereinafter referred to as “mouse fibroblast proliferation evaluation medium”), where the mouse fibroblast was cultured for 12 hours in 5% CO₂ and saturated water vapor atmosphere at 37° C.

### (2) Addition of Saturated and Unsaturated Fatty Acids

When investigating the effects of saturated and unsaturated fatty acids on the proliferation of the fibroblast, 5 μM and 10 μM palmitic acid (S-33, Wako Pure Chemical Industries, Ltd.), 5 μM and 10 μM oleic acid (07501, Sigma Aldrich Japan K.K.) or 1 μM, 3 μM and 10 μM stearic acid (S3381, Sigma Aldrich Japan. K.K.) was added after 12 hours to the mouse fibroblast proliferation evaluation medium), and the fibroblast was cultured for 48 hours in 5% CO₂ and saturated water vapor atmosphere at 37° C. As a control, the cell was cultured in a culture medium without these saturated fatty acids and unsaturated fatty acid.

### (3) Determination of % of Proliferation of Fibroblast

Thereafter, alamar Blue (Trade mark, DAL1025, Invitrogen, Life Technologies Japan Ltd.) was added to the culture medium at a final concentration of 10%, and its supernatant was examined for the fluorescent intensity 2 hours later with an excitation wavelength of 544 nm and a fluorescent intensity of 590 nm in accordance with the manufacture's instruction. The cell proliferation rate (%) was calculated as a percentage of the quotient obtained by dividing the fluorescent intensity of the alamar Blue under each experiment condition by the fluorescent intensity in the control group supplemented with no saturated or unsaturated fatty acids.

### 2. Results

### (1) Addition of Palmitic Acid

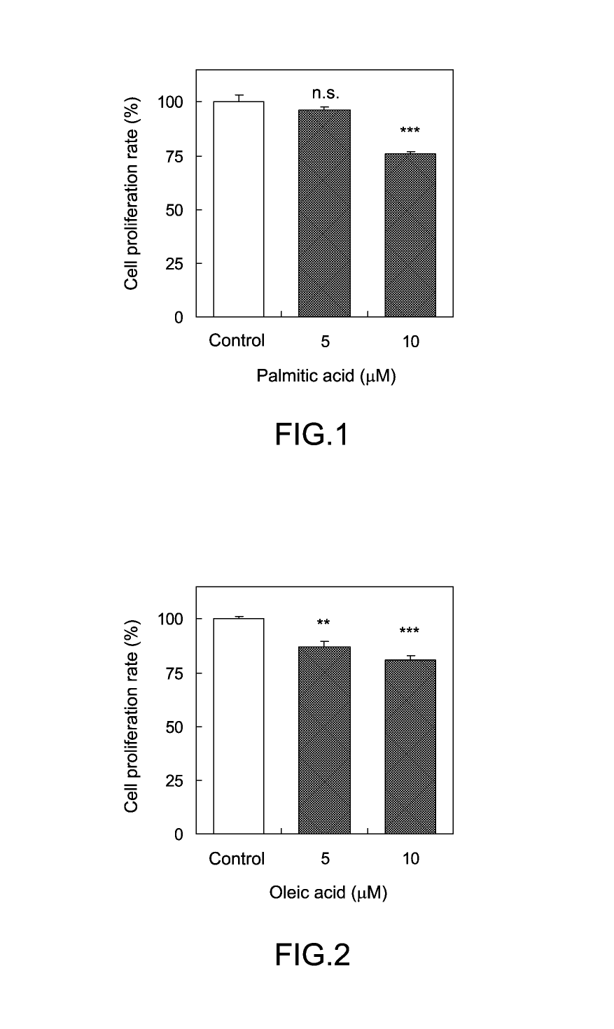

FIG. 1 is a graph showing the results of the experiment investigating the effect of palmitic acid on the mouse fibroblast proliferation. The error bars for relevant experimental conditions are the standard errors of the measured values of the experimental results in which the cell proliferation in 3 wells in each of the control group and the treatment group was repeatedly measured several times under identical conditions. The asterisk (***) indicates p<0.1% in Fisher's PLSD test. The indication “n.s.” in the graph refers to no significant difference when compared with the control. As shown in FIG. 1, the addition of palmitic acid at 10 μM resulted in a statistically significant reduction in the cell proliferation.

### (2) Addition of Oleic Acid

FIG. 2 is a graph showing the results of the experiment investigating the effect of oleic acid on the mouse fibroblast proliferation. The error bars for relevant experimental conditions are the standard errors of the measured values of the experimental results in which the cell proliferation in 3 wells in each of the control group and the treatment group was repeatedly measured several times under identical conditions. The asterisk (***) indicates p<0.1% and the asterisk (**) indicates p<1% in Fisher's PLSD test. As shown in FIG. 2, the addition of oleic acid at 5 μM and 10 μM resulted in a statistically significant reduction in the cell proliferation.

### (3) Stearic Acid Addition

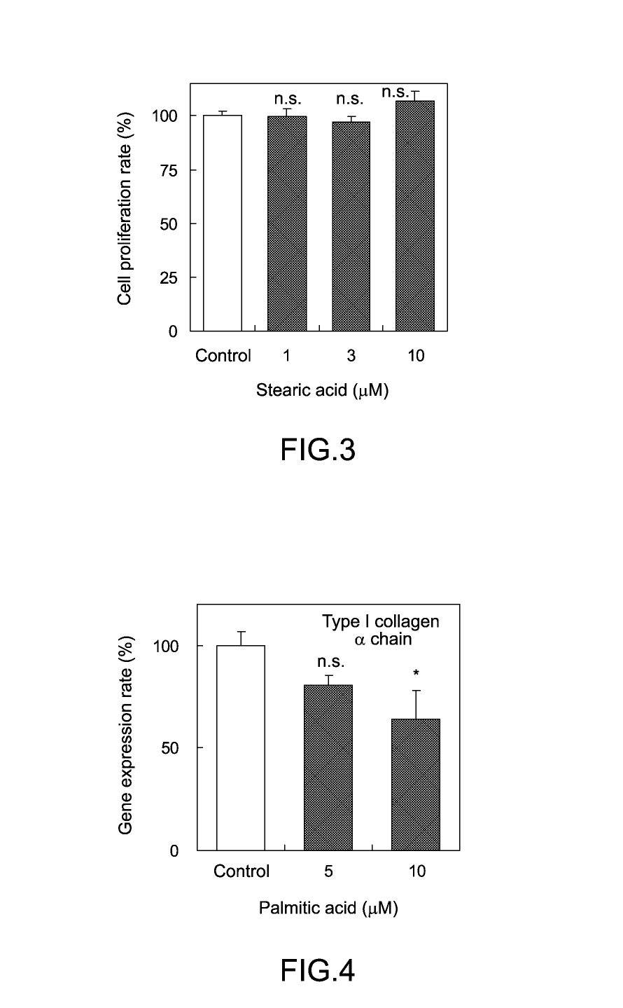

FIG. 3 is a graph showing the results of the experiment investigating the effect of stearic acid on the mouse fibroblast proliferation. The error bars for relevant experimental conditions are the standard errors of the measured values of the experimental results in which the cell proliferation in 3 wells in each of the control group and the treatment group was repeatedly measured several times under identical conditions. The indication “n.s.” in the graph refers to no significant difference when compared with the control. As shown in FIG. 3, the addition of stearic acid at 1 μM to 10 μM resulted in no statistically significant reduction in the cell proliferation.

### 3. Conclusion

Based on the experimental results in this Example, effect of supplementing the mouse fibroblast proliferation was observed when using palmitic acid and oleic acid, and was not observed when using stearic acid. Accordingly, it is suggested that a reduction in the amount of palmitic and oreic acids can alleviate the suppression of the fibroblast proliferation.

### Example 2

### Effect of Palmitic Acid on Gene Expression in Mouse Fibroblast

### 1. Materials and Methods

The cell culture and the addition of palmitic acid were conducted similarly to Example 1. The mouse fibroblast was inoculated to and incubated in a commercially available 12-well culture plate (353043, FALCON, Becton, Dickinson and Company Japan) at 3×10⁴ cells/mL.

### Quantification of Type I Collagen α Chain, Elastin and Matrix Metalloproteinase 13 Gene Expressions

The culture medium was removed using an aspirator, and each well was washed twice with each 2 ml of PBS. An RNA was extracted from each cell in each well using an RNeasy Protect Kit (74104, Qiagen) according to Manufacturer's instructions. A cDNA was produced according to a standard method and used in a real-time PCR. This real-time PCR employed LightCycler (registered trademark) FastStart DNA Master^{PLUS} SYBR Green I (Catalog No. 03 515 885 001, Roche Diagnostic K.K.). For amplifying type I collagen α chain, elastin, matrix metalloproteinase 13 and 28S rRNA genes, the forward and reverse primers of SEQ ID NO: 1 and 2, SEQ ID NO: 3 and 4, SEQ ID NO: 5 and 6 and SEQ ID NO: 7 and 8, respectively, were employed. The PCR was conducted under a reaction condition involving 1 cycle of 95° C. for 10 minutes and 35 cycles of 95° C. for 15 seconds, 60° C. for 5 seconds and 72° C. for 5 seconds. The expression levels of type I collagen α chain, elastin and matrix metalloproteinase 13 gene were analyzed by using LightCycler Software Ver. 3.5 (Roche Diagnostic K.K.) and normalized on the basis of the expression level of 28S rRNA. The gene expressions level of the type I collagen α chain, elastin and matrix metalloproteinase 13 were calculated as percentages of the quotients obtained by dividing the gene expression rates (%) under each experimental condition by the gene expression levels in the control groups containing no palmitic acid.

### 2. Results

### (1) Expression of Type I Collagen α Chain

FIG. 4 is a graph showing the results of the experiment investigating the effect of palmitic acid on the type I collagen α chain gene expression in the mouse fibroblast. The error bars for relevant experimental conditions are the standard errors of the measured values of the experimental results in which the gene expression in 3 wells in each of the control group and the treatment group was repeatedly measured several times under identical conditions. The asterisk (*) indicates p<5% in Fisher's PLSD test. The indication “n.s.” in the graph refers to no significant difference when compared with the control. As shown in FIG. 4, the addition of palmitic acid at 10 μM resulted in a statistically significant reduction in the type I collagen α chain gene expression.

### (2) Elastin Expression

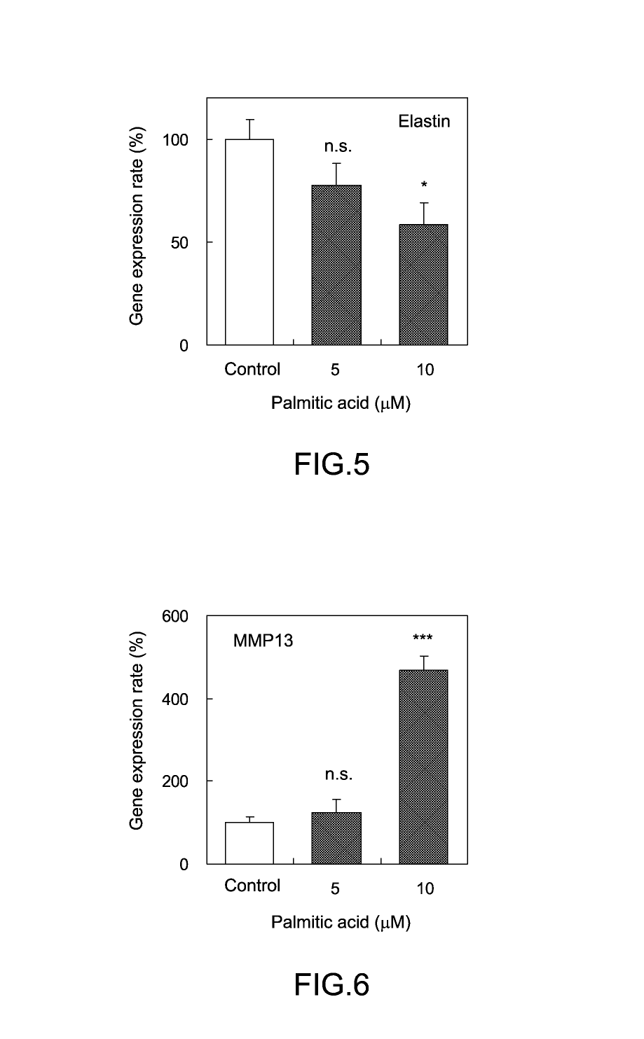

FIG. 5 is a graph showing the results of the experiment investigating the effect of palmitic acid on the elastin gene expression in the mouse fibroblast. The error bars for relevant experimental conditions are the standard errors of the measured values of the experimental results in which the gene expression in 3 wells in each of the control group and the treatment group was repeatedly measured several times under identical conditions. The asterisk (*) indicates p<5% in Fisher's PLSD test. The indication “n.s.” in the graph refers to no significant difference when compared with the control. As shown in FIG. 5, the addition of palmitic acid at 10 μM resulted in a statistically significant reduction in the elastic gene expression.

### (3) Matrix Metalloproteinase 13 Expression

FIG. 6 is a graph showing the results of the experiment investigating the effect of palmitic acid on the matrix metalloproteinase 13 gene expression in the mouse fibroblast. The error bars for relevant experimental conditions are the standard errors of the measured values of the experimental results in which the gene expression in 3 wells in each of the control group and the treatment group was repeatedly measured several times under identical conditions. The asterisk (***) indicates p<0.1% in Fisher's PLSD test. The indication “n.s.” in the graph refers to no significant difference when compared with the control. As shown in FIG. 6, the addition of palmitic acid at 10 μM resulted in a statistically significant increase in the matrix metalloproteinase gene expression.

### 3. Conclusion

Based on the experimental results in this Example, palmitic acid had a suppressive effect on the gene expression of type I collagen α chain and elastic, and a promotive effect on the gene expression of matrix metalloproteinase 13. On the other hand, oleic acid and stearic acid did not have a suppressive effect on the gene expression of type I collagen α chain and elastin, and a promotive effect on the gene expression of matrix metalloproteinase 13 (data not shown).

### Example 3

### Human Fibroblast Proliferation Suppressing Effect of Palmitic Acid

### 1. Materials and Methods

The cell culture, the addition of palmitic acid and the determination of the % of proliferation of the fibroblast were conducted similarly to Example 1. The human fibroblast (CC-2509, Lonza Japan Ltd.) was incubated for 6 hours in a commercially available cell culture medium (D-MEM, 11885084, GIBCO, Life Technologies Japan Ltd.) supplemented with 10% of fetal bovine serum (FBS) (Biowest). Thereafter, the culture medium for incubating the human fibroblast was switched to the cell culture medium supplemented with 0.5% of fetal bovine serum (hereinafter referred to as “human fibroblast proliferation evaluation medium”) and the human fibroblast was incubated at 37° C. and 5% CO₂ under a saturated water vapor atmosphere for 12 hours. Palmitic acid (S-33, Wako Pure Chemical Industries, Ltd.) was added at 5 μM, 10 μM, 20 μM and 30 μM to the human fibroblast proliferation evaluation medium after incubating for 12 hours.

### 2. Results

### Addition of Palmitic Acid

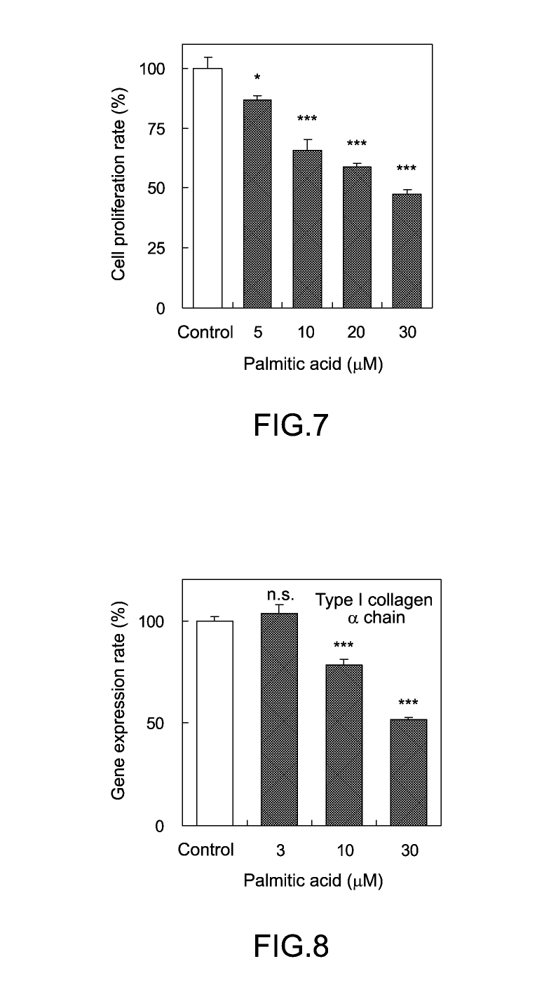

FIG. 7 is a graph showing the results of the experiment investigating the effect of palmitic acid on the human fibroblast proliferation. The error bars for relevant experimental conditions are the standard errors of the measured values of the experimental results in which the cell proliferation in 3 wells in each of the control group and the treatment group was repeatedly measured several times under identical conditions. The asterisk (*) indicates p<5% and the asterisk (***) indicates p<0.1% in Fisher's PLSD test. As shown in FIG. 7, the addition of palmitic acid at 5 μM to 30 μM resulted in a statistically significant reduction in the cell proliferation which was dependent on the concentration of the added palmitic acid. Based on the results of this Example, the proliferation suppressing effect of palmitic acid was observed not only in the mouse fibroblast but also in the human fibroblast

### Example 4

### Effect of Palmitic Acid on Gene Expression in Human Fibroblast

The cell culture and the addition of palmitic acid were conducted similarly to Example 3. Palmitic acid was added at 3 μM, 10 μM and 30 μM. The quantification of gene expression was conducted similarly to Example 2. For amplifying type I collagen α chain, elastin and matrix metalloproteinase 1, the forward and reverse primers of SEQ ID NO: 9 and 10, SEQ ID NO: 11 and 12 and SEQ ID NO: 13 and 14, respectively, were employed. The % gene expressions of the type I collagen α chain, elastin and matrix metalloproteinase 1 were calculated as percentages of the quotients obtained by dividing the gene expression levels under each experimental condition by the gene expression levels in the control groups containing no palmitic acid.

### 2. Results

### (1) Type I Collagen α Chain Expression

FIG. 8 is a graph showing the results of the experiment investigating the effect of palmitic acid on the type I collagen α chain gene expression in the human fibroblast. The error bars for relevant experimental conditions are the standard errors of the measured values of the experimental results in which the gene expression in 3 wells in each of the control group and the treatment group was repeatedly measured several times under identical conditions. The asterisk (***) indicates p<0.1% in Fisher's PLSD test. The indication “n.s.” in the graph refers to no significant difference when compared with the control. As shown in FIG. 8, the addition of palmitic acid at 10 μM and 30 μM resulted in a statistically significant reduction in the type I collagen α chain gene expression which was dependent on the concentration of the added palmitic acid.

### (2) Expression of Elastin

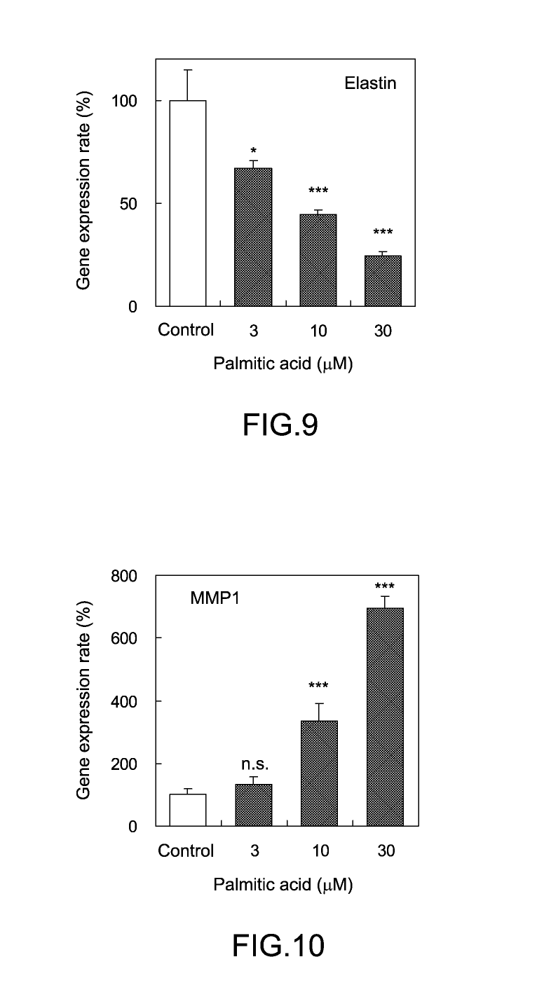

FIG. 9 is a graph showing the results of the experiment investigating the effect of palmitic acid on the elastin gene expression in the human fibroblast. The error bars for relevant experimental conditions are the standard errors of the measured values of the experimental results in which the gene expression in 3 wells in each of the control group and the treatment group was repeatedly measured several times under identical conditions. The asterisk (*) indicates p<5% and the asterisk (***) indicates p<0.1% in Fisher's PLSD test. As shown in FIG. 9, the addition of palmitic acid at 3 μM to 30 μM resulted in a statistically significant reduction in the elastin gene expression which was dependent on the concentration of the added palmitic acid.

### (3) Expression of Matrix Metalloproteinase 1

FIG. 10 is a graph showing the results of the experiment investigating the effect of palmitic acid on the matrix metalloproteinase 1 gene expression in the human fibroblast. The error bars for relevant experimental conditions are the standard errors of the measured values of the experimental results in which the gene expression in 3 wells in each of the control group and the treatment group was repeatedly measured several times under identical conditions. The asterisk (***) indicates p<0.1% in Fisher's PLSD test. The indication “n.s.” in the graph refers to no significant difference when compared with the control. As shown in FIG. 10, the addition of palmitic acid at 10 μM and 30 μM resulted in a statistically significant increase in the matrix metalloproteinase 1 gene expression which was dependent on the concentration of the added palmitic acid.

### 3. Conclusion

Based on the experimental results in this Example, palmitic acid had a suppressive effect on the gene expression of type I collagen α chain and elastin, and a promotive effect on the gene expression of matrix metalloproteinase 1. Accordingly, it was suggested that the skin elastic property loss is caused by an increase in palmitic acid in a vital body. It is also suggested that an aggravation of a skin condition due to a skin elastic property loss can be prevented and/or improved by reducing the palmitic acid level.

### Example 5

### Effect of EPA on Fibroblast Proliferation

### 1. Materials and Methods

The cell culture, the addition of saturated fatty acids and unsaturated fatty acids and the determination of the % of proliferation of the fibroblast were conducted similarly to Example 1. In the addition of saturated fatty acids and unsaturated fatty acids, eicosapentaenoic acid (EPA) (14326-04, Nacalai Tesque, Inc.) at 0.5 μM, palmitic acid at 10 μM and a mixture of palmitic acid at 10 μM and EPA at 0.5 μM were added. The cell culture in a medium containing no such saturated fatty acids and unsaturated fatty acids served as a control.

### 2. Results

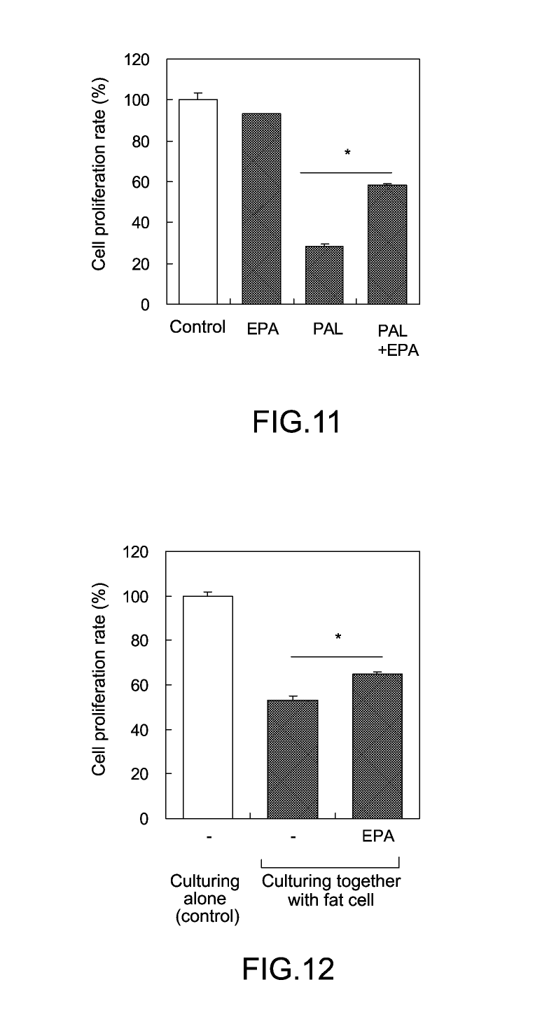

FIG. 11 is a graph showing the results of the experiment investigating the effect of palmitic acid on the mouse fibroblast proliferation. The error bars for relevant experimental conditions are the standard errors of the measured values of the experimental results in which the cell proliferation in 3 wells in each of the control group and the treatment group was repeatedly measured several times under identical conditions. The asterisk (*) indicates p<5% in Fisher's PLSD test. As shown in FIG. 11, the cell proliferation was reduced markedly by the addition of palmitic acid (PAL) but not by the EPA addition. When adding the mixture (PAL+EPA), the cell proliferation was not reduced markedly in spite of the addition of the palmitic acid at the concentration similar to that in the addition of the palmitic acid (PAL). From the results of the experiment in this Example, it was indicated that EPA has an antagonistic effect on palmitic acid and results in a statistically significant alleviation of the fibroblast proliferation suppression by palmitic acid. It was also suggested that a polyvalent unsaturated fatty acid EPA inhibits the physiological activity of palmitic acid in a vital body, and is effective in preventing and/or improving a skin elastic property loss and an aggravation of a skin condition due to such a skin elastic property loss.

### Example 6

### Effect of Fat Cell and EPA on Fibroblast Proliferation During Culturing Together

### 1. Materials and Methods

A mouse 3T3-L1 cell was employed as a fibroblast and the fat cell induced differentiation from the 3T3-L1 cell during culture was employed as a fat cell. The 3T3-L1 cell was grown in a Dulbecco's Modified Eagle Medium (DMEM) supplemented with 10% fetal bovine serum (FBS). The differentiation induction of the 3T3-L1 cell was conducted as described below. Thus, a 6-well multiwell plate for culturing together (Cell Culture Insert/Companion plate, FALCON, Becton, Dickinson and Company Japan) was inoculated with 7.5×10⁴ (7.5×10 sup. 4) 3T3-L1 cells per well, which was then incubated for two days at 37° C. in a DMEM supplemented with 10% FBS containing insulin, dexamethasone and isobutylmethyl xanthine (at final concentrations of 0.2, 0.3 and 200 micromoles, respectively). Thereafter, the cells were incubated for two days at 37° C. in DMEM supplemented with 10% FBS containing only insulin (0.2 micromoles). The 3T3-L1 cells after the differentiation induction were incubated at 37° C. in DMEM supplemented with 10% FBS. The 3T3-L1 cell induced differentiation was subjected to culturing together with the 3T3-L1 fibroblast which was not induced to differentiate on the 21st day after initiation of the differentiation induction. The 3T3-L1 fibroblast incubated in DMEM supplemented with 10% FBS without differentiation induction was inoculated in a container hanged in the wells (Cell Culture insert through which the cell cannot permeate but the culture medium components can permeate; 1.0 μm (micrometer) in pore size, 1.6×10⁶ pores/cm² (6×10 .sup. 6 pores/cm .sup. 2) in pore density, FALCON, Becton, Dickinson and Company Japan) at 3×10⁴ (3×10 .sup. 4) 3T3-L1 cells per well. The fat cell and the fibroblast were both subjected to culturing together 12 hours after switching into DMEM supplemented with 0.5% FBS. When investigating the effect of EPA while culturing the fibroblast and the fat cell together, 1 μM of EPA was added to the DMEM supplemented with 0.5% FBS. In the control experiment, the fibroblast inoculated in the container was incubated alone in a well containing DMEM supplemented with 0.5% FBS. After the culturing together for two days, the fibroblast in the container was recovered and the cell proliferation rate (%) was determined similarly to Example 1 using alamar Blue.

### 2. Results

FIG. 12 is a graph showing the effect of the fat cell and EPA on the fibroblast proliferation during culturing together. The error bars for relevant experimental conditions are the standard errors of the measured values of the experimental results in which the cell proliferation in 3 wells in each of the control group and the treatment group was repeatedly measured several times under identical conditions. The asterisk (*) indicates p<5% in Fisher's PLSD test. As shown in FIG. 12, the fat cell markedly suppressed the fibroblast proliferation, while EPA resulted in a statistically significant decrease in the fibroblast proliferation suppression by the fat cell. Based on the experimental results in this Example, it was suggested that a reduction in the fibroblast and the extracellular matrix component in a dermis is caused by an increase in the palmitic acid level in a vital body attributable to an increased and/or hypertrophic fat cell in a subcutaneous tissue.

It was suggested that by reducing the fat cell count and the palmitic acid level or by inhibiting the physiological activity of palmitic acid, a skin elastic property loss and an aggravation of a skin condition, such as wrinkling, sagging, bedsore and wound healing disorder due to such a skin elastic property loss, can be prevented and/or improved.

We claim:

1. A method for preventing and improving a skin elastic property loss, comprising a step of suppressing an increase in palmitic acid or a step of inhibiting the bioactivity of palmitic acid.

2. The method for preventing and improving a skin elastic property loss according to claim 1, wherein the step of suppressing an increase in palmitic acid comprises a step of administering a composition which suppresses an increase in palmitic acid to a subject.

3. The method for preventing and improving a skin elastic property loss according to claim 1, wherein the step of inhibiting the bioactivity of palmitic acid comprises a step of administering a composition which inhibits the bioactivity of palmitic acid to a subject.

4. The method for preventing and improving a skin elastic property loss according to claim 3, wherein the composition which inhibits the bioactivity of palmitic acid is a composition comprising one or two or more of compounds selected from the group consisting of a polyvalent unsaturated fatty acid and a derivative and/or a salt thereof.

5. The method for preventing and improving a skin elastic property loss according to claim 4, wherein the polyvalent unsaturated fatty acid is eicosapentaenoic acid.

6. A cosmetic method for preventing and improving wrinkling and sagging accompanied with a skin elastic property loss, comprising a step of applying to a skin a method for preventing and improving a skin elastic property loss according to claim 1.

7. A method for preventing and treating a wound healing disorder and a bedsore accompanied with a skin elastic property loss, comprising a step of applying to a skin a method for preventing and improving a skin elastic property loss according to claim 1.

8. A method for preventing and improving a reduction in an extracellular matrix component, comprising a step of suppressing an increase in palmitic acid or a step of inhibiting the bioactivity of palmitic acid.

9. The method for preventing and improving a reduction in an extracellular matrix component according to claim 8, wherein the step of suppressing an increase in palmitic acid comprises a step of administering a composition which suppresses an increase in palmitic acid to a subject.

10. The method for preventing and improving a reduction in an extracellular matrix component according to claim 8, wherein the step of inhibiting the bioactivity of palmitic acid comprises a step of administering a composition which inhibits the bioactivity of palmitic acid to a subject.

11. The method for preventing and improving a reduction in an extracellular matrix component according to claim 10, wherein the composition which inhibits the bioactivity of palmitic acid is a composition comprising one or two or more of compounds selected from the group consisting of a polyvalent unsaturated fatty acid and a derivative and/or a salt thereof.

12. The method for preventing and improving a reduction in an extracellular matrix component according to claim 11, wherein the polyvalent unsaturated fatty acid is eicosapentaenoic acid.

13. The method for preventing and improving a reduction in an extracellular matrix component according to claim 8, wherein the extracellular matrix component is a collagen and/or an elastin.

14. A cosmetic method for preventing and improving wrinkling and sagging, comprising a step of applying to a skin a method for preventing and improving a reduction in an extracellular matrix component according to claim 8.

15. A method for preventing and treating a wound healing disorder and a bedsore, comprising a step of applying to a skin a method for preventing and improving a reduction in an extracellular matrix component according to claim 8.

16. The method for preventing and improving according to claim 1, wherein the palmitic acid is produced by a hypertrophic fat cell.

17. The method for preventing and improving according to claim 8, wherein the palmitic acid is produced by a hypertrophic fat cell.
